(19) United States
(12) Patent Application Publication
(10) Pub. No.: US 2021/0354122 A1
(43) Pub. Date: Nov. 18, 2021

(54) SYSTEM AND CARTRIDGE FOR SAMPLE TESTING

(71) Applicant: Quidel Corporation, San Diego, CA (US)

(72) Inventors: Johannes Kehle, San Diego, CA (US); Bruce Jacono, San Diego, CA (US); Werner Kroll, Wayland, MA (US); Kunal Sur, San Diego, CA (US); Zaheer Parpia, San Diego, CA (US); Andrew Eliopoulos, San Diego, CA (US); Matthew Morovich, San Diego, CA (US); Jesse McDaniels-Davidson, San Diego, CA (US); Jon Bjarnason, San Diego, CA (US); Jesse Witkowicki, San Diego, CA (US); Todd Linhoff, San Diego, CA (US); Todd Denison Pack, San Diego, CA (US)

(21) Appl. No.: 17/319,946

(22) Filed: May 13, 2021

Related U.S. Application Data
(60) Provisional application No. 63/024,406, filed on May 13, 2020.

(51) Int. Cl. B01L 3/00; C12Q 1/6806; C12N 15/10; C12Q 1/6848; G01N 21/55

(52) U.S. Cl. CPC B01L 3/502; B01L 3/527; B01L 3/561; C12Q 1/6806; C12N 15/1013; C12Q 1/6848; B01L 2200/141; B01L 2300/044; B01L 2300/0609; B01L 2300/0663; B01L 2200/10; B01L 2200/16; G01N 21/55

(57) ABSTRACT

A system for analysis of a sample to aid in diagnosis and/or detection of the presence or absence, and/or the identity of, an analyte in the sample is provided. A cartridge with reagents to isolate nucleic acid from a sample inserted in the cartridge and to amplify the isolated nucleic acid, and an instrument that interacts with the cartridge, together provide a self-contained sample to answer system for detection, identification, differentiation and/or quantification of a target nucleic acid in a sample.

### CROSS REFERENCE TO RELATED APPLICATIONS

This application claims priority to, and benefit of, U.S. Provisional Application No. 63/024,406, filed May 13, 2020, the entire content of which is hereby incorporated by reference.

### TECHNICAL FIELD

The subject matter described herein relates to a system for analysis of a sample to, for example, detect the presence or absence of a target analyte in the sample, and/or to determine the identity of an analyte in the sample. The system, for example, aids in the determination or diagnosis of a condition, disease or disorder due to the presence of an analyte, such as an infectious agent, in the sample. The system comprises a cartridge and an instrument that receives the cartridge, and that function together to provide analysis of a sample inserted into the cartridge, for detection, identification, differentiation and/or quantification of the presence of a target nucleic acid in a sample.

### BRIEF DESCRIPTION

In the analysis of a sample, determining presence of certain nucleic acids is desirable for a high level of accuracy and sensitivity. The availability of amplification techniques, such as polymerase chain reaction (PCR) and other nucleic acid amplification technologies, make nucleic acid detection and differentiation a sensitive technique for analysis of a pathogen or other agent in a sample of biological origin. However, the need to perform multiple reagent steps and to handle the sample and perform the analysis in a manner to avoid contamination in order to achieve accuracy of the analysis hinder the broad application of these techniques, outside of the sophisticated microbiology laboratory or clinical settings.

### BRIEF SUMMARY

In a first aspect, a system comprising a cartridge and an instrument is provided. The cartridge comprises (i) a plurality of chambers, wherein the plurality of chambers includes an extraction chamber and a detection chamber; (ii) a plurality of reagent canisters, each reagent canister in the plurality including a reagent; and (iii) a plurality of magnetic particles which once introduced into the extraction chamber, are retained therein. The instrument is configured to receive the cartridge and is comprised of (i) a first sonicator movable in at least one of the x-y-z coordinates; (ii) a magnetic field positionable to capture the plurality of magnetic particles in the extraction chamber; and (iii) an optical unit for illumination of the detection chamber and for detection of a signal therefrom.

In an embodiment, a cartridge comprises (i) a plurality of chambers, wherein the plurality of chambers includes an extraction chamber and at least one detection chamber; (ii) a port for engagement with a gas supply source; (iii) a plurality of reagent canisters, each reagent canister in the plurality including a reagent; and (iv) a plurality of magnetic particles. In some embodiments, all or a portion of the reagent canisters may be in fluid communication with a dedicated gas source via one or more of the gas supply ports. In an embodiment, the piercing element may be in fluid communication with a dedicated gas source via one or more of the gas supply ports. In some embodiments, the magnetic particles are in a first location on the cartridge and movable into the extraction chamber. In another embodiment, the magnetic particles are in the extraction chamber prior to use of the cartridge and remain in the extraction chamber after use, when the cartridge is disposed. In another embodiment, the magnetic particles are in a first location on the cartridge and movable into the extraction chamber in conjunction with transfer of a fluid comprising the sample, that is inserted into a sample port on the cartridge, when the sample-containing fluid is moved into the extraction chamber.

In an embodiment, each reagent canister comprises a frangible material and comprises, or is configured for contact with, a piercing element. The piercing element, in an embodiment, is a component of the reagent canister. In other embodiments, the piercing element is a component of the cartridge or the instrument, and is positioned for contact with the frangible material on the reagent canister. In an embodiment, the piercing element comprises an opening capable of fluid communication with the reagent canister and/or a conduit connecting the reagent canister to the cartridge. In some embodiments, the opening of the piercing element defines a conduit with an inlet and an outlet. In use, a gas via a gas supply port on the cartridge is introduced into the inlet of the piercing element to displace a reagent in the reagent canister for transfer via the outlet and into the cartridge.

In an embodiment, the cartridge comprises one port or a plurality of ports, configured for engagement with the gas supply source. In another embodiment, the gas supply source is a pressurized gas source contained within the instrument that receives the cartridge. In other embodiments, the gas supply source is a pressurized gas source external to the instrument and the system.

In another aspect, a method for identifying presence or absence of a target nucleic acid (i.e., detection, identification, and/or differentiation) in a sample is provided. The method comprises (i) providing a cartridge including an extraction chamber, a detection chamber disposed downstream from the extraction chamber; (ii) moving a sample suspected of including a target nucleic acid sample, a plurality of magnetic particles, and a fluid into the extraction chamber; (iii) capturing the plurality of magnetic particles complexed with the nucleic acid with a magnetic field and, with the extraction chamber essentially or substantially empty of fluid, introducing gas at a temperature of above about 35° C. into the extraction chamber; (iv) introducing a volume of an elution medium into the extraction chamber; (v) releasing the plurality of magnetic particles complexed with nucleic acid from the magnetic field into the elution medium to release the nucleic acid from the plurality of magnetic particles; (vi) capturing the plurality of magnetic particles with a magnetic field to retain the plurality of magnetic particles in the extraction chamber; (vii) moving the elution medium and the nucleic acid from the extraction chamber into a downstream chamber for contact with reagents for amplification of the nucleic acid; and (viii) amplifying the nucleic acid and detecting in the detection chamber the amplification products.

In another aspect, a kit comprising a cartridge and a pipette is provided. In some embodiments, the pipette comprises an overflow chamber. In other embodiments, the pipette is configured to dispense a specific fixed and known volume of a sample, such as a patient sample, into the cartridge. In embodiments, the cartridge comprises reagents for isolating a target nucleic acid from the sample, and amplifying the target nucleic acid (if present). The cartridge is insertable into an instrument which is configured to detect presence (or absence) if amplicons of the target nucleic acid and report a result to a user of the cartridge.

### BRIEF DESCRIPTION OF THE FIGURES

FIG. 1 illustrates a testing architecture or system composed of an instrument, an optical source and a cartridge, according to some embodiments.

FIGS. 2A-2D illustrate different views of a cartridge for sample testing, according to some embodiments.

FIG. 3 illustrates a chamber and a mechanism for unloading a canister including reagents in a cartridge for sample testing, according to some embodiments.

FIG. 4 illustrates a valve used to control fluid flow in a cartridge for sample testing, the indicated dimensions having units of mm, according to some embodiments.

FIG. 5 illustrates a partial view of a detection chamber panel in a cartridge for sample testing, according to some embodiments.

FIGS. 6A-6D illustrate components in an optical coupler for use in an optical unit for an instrument, according to some embodiments.

FIGS. 7A-7AF illustrate a sequence of steps in a sample testing method using a cartridge, according to some embodiments.

FIG. 8 is a flow chart illustrating steps in a sample testing method, according to some embodiments.

In the figures, elements with the same or similar labels may refer to elements having the same or similar features, unless otherwise stated.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

In the field of nucleic acid detection, the ability to amplify target samples at an exponential rate using techniques, such as polymerase chain reaction (PCR) or an isothermal process such as helicase dependent amplification, has significantly increased the sensitivity of detection. However, the delicate balance of multiple reagent steps without contamination has prevented the application of these techniques in compact, easy to use instruments that produce accurate results in a limited amount of time.

The systems, assays, devices, methods and kits described herein, can be used for qualitative detection and/or differentiation of various and multiple analytes, such as target nucleotides which may be associated with, for example, a pathogen, microbe, bacteria, virus, fungus or other microorganism. For example, in some embodiments the present technology is related to a rapid multiplexed Real-Time PCR (RT-PCR) assay for the qualitative detection and differentiation of a nucleotide from a pathogen of interest. In some embodiments, pathogens of interest may include microbes, bacteria, virus, fungus or other microorganisms and infectious agents. In one embodiment, the pathogen of interest is a virus, such as, influenza A (Flu A), influenza B (Flu B), respiratory syncytial virus (RSV), or SARS-CoV-2.

The systems, assays, devices, methods and kits described herein, provide analysis of various nucleic acids of interest, such as DNA and/or RNA. In some embodiments, the nucleic acid of interest is a viral RNA extracted from nasal and nasopharyngeal swab in viral transport media. In some embodiments, analyzed specimens, including nasal and nasopharyngeal swabs, are from patients with signs and symptoms of respiratory viral infection.

The systems, assays, devices, methods and kits described herein, also provide, in some embodiments, flexibility for users to choose what results, such as identification of the presence or absence of a viral RNA, may be reported. Accordingly, in some embodiments, the systems, assays, devices, methods and kits described herein, provide in vitro diagnostic tests intended to aid in the differential diagnosis of diseases, such as viral diseases. In some embodiments, the viral diseases of interest include, but are not limited to, Flu A, Flu B, RSV and SARS-CoV-2. The technology provided herein can provide information related to infection, such as viral infection, in humans in conjunction with clinical and epidemiological risk factors.

In some embodiments, the technology provided herein involves testing that is performed laboratory personnel, such as personnel in laboratories certified under the Clinical Laboratory Improvement Amendments of 1988 (CLIA), 42 U.S.C. § 263a, to perform moderate/high complexity tests. In other embodiments, the systems, assays, devices, methods and kits described herein can be distributed and used in other settings, such as patient care settings outside of the clinical laboratory environment.

In some embodiments, the systems, assays, devices, methods and kits described herein, provide results indicating a positive or negative result for detection of a pathogen of interest, such as a bacteria, virus, fungus or other microbe or microorganism. In some embodiments, positive or negative results may be indicative of the presence of a viral infection, such the presence of Flu A, Flu B, RSV or SARS-CoV-2. In some embodiments, positive or negative results provided by the technology provided herein may be considered in coordination with clinical correlation of patient history and other diagnostic information that may be necessary to determine patient infection status. For example, a positive or negative result for one pathogen, such as a viral pathogen, does not rule out the possibility of additional infections, such as bacterial infections or co-infection with other viruses.

In some embodiments, the technology provided herein may provide information related to infection with novel pathogens, such as, for example, a novel influenza virus. In such instances, specimens should be collected and handled according to proper safety, documentation and submission guidelines.

In some embodiments, the systems, assays, devices, methods and kits described herein, provide determination of the presence or absence of influenza viral nucleic acids in a sample, such as a nasal or nasopharyngeal swab from a patient suspected of pathogenic infection and/or disease. Influenza viruses are causative agents of highly contagious, acute, viral infections of the respiratory tract. Influenza viruses are immunologically diverse, single-stranded RNA viruses. There are three types of influenza viruses: A, B, and C. Type A viruses are the most prevalent and are associated with most serious epidemics. Type B viruses produce a disease that is generally milder than that caused by type A. Type C viruses have never been associated with a large epidemic of human disease. Both Type A and B viruses can circulate simultaneously, but usually one type is dominant during a given season. Every year in the United States, on average 5%-20% of the population contract influenza; more than 200,000 people are hospitalized from influenza complications; and, about 36,000 people die from influenza-related causes. Some people, such as adults 65 years of age and older, young children, and people with certain health conditions, are at high risk for serious influenza complications.

In some embodiments, the systems, assays, devices, methods and kits described herein, provide determination of the presence or absence of SARS-CoV-2 virus nucleic acids in a sample, such as a nasal or nasopharyngeal swab from a patient suspected of pathogenic infection and/or disease. SARS-CoV-2, also known as the COVID-19 virus, was first identified in Wuhan, Hubei Province, China December 2019. This virus, as with the novel coronavirus SARS-1 and MERS, is thought to have originated in bats, however the SARS-CoV-2 may have had an intermediary host such as pangolins, pigs or civets. The WHO declared that COVID-19 was a pandemic on Mar. 11, 2020, and human infection spread globally, with hundreds of thousands of confirmed infections and deaths. The median incubation time is estimated to be 5.1 days with symptoms expected to be present within 12 days of infection. The symptoms of COVID-19 are similar to other viral respiratory diseases and include fever, cough and shortness of breath.

In some embodiments, the systems, assays, devices, methods and kits described herein, provide determination of the presence or absence of Human respiratory syncytial virus (RSV) nucleic acids in a sample, such as a nasal or nasopharyngeal swab from a patient suspected of pathogenic infection and/or disease. RSV, is a negative single-stranded RNA virus of the family Paramyxoviridae. RSV is the major cause of lower respiratory tract infection and hospital visits during infancy and childhood. In the United States, 60% of infants are infected during their first RSV season, and nearly all children will have been infected with the virus by 2-3 years of age. Of those infected with RSV, 2-3% will develop bronchiolitis, necessitating hospitalization. Natural infection with RSV induces protective immunity that wanes over time—possibly more so than other respiratory viral infections—and thus people can be infected multiple times. Sometimes an infant can become symptomatically infected more than once, even within a single RSV season. Severe RSV infections have increasingly been found among elderly patients.

In some embodiments, the systems, assays, devices, methods and kits described herein provide an approach for a single, disposable, self-contained assay cartridge with reagents for a nucleic acid amplification process, such as real time PCR or other amplification technology, that is used in conjunction with an instrument to detect and differentiate target nucleotides in a sample that is inserted into the cartridge. In some embodiments, the target nucleotides that may be detected with the system, cartridge, and methods provided herein are nucleotides from pathogens and/or microbes, such as bacteria, virus or fungus. For example, in some embodiments, the analyzed, detected and/or differentiated nucleotides may comprise DNA and/or RNA, such as RNA from influenza A, influenza B, RSV, and/or SARS-CoV-2. Other exemplary target nucleotide analytes include those from Bordetella pertussis, Brodetella parapertussis, C. difficile, Group A β-hemolytic Streptococcus (Streptococcus pyogenes), pyogenic Group C/G (Streptococcus dysgalactiae), herpes simplex virus 1, herpes simplex virus 2, varicella-zoster virus, human metapneumovirus, trichomonas, human adenovirus, and parainfluenza virus (PIV-1, PIV-2, and/or PIV-3). The self-contained cartridge can comprise reagents for detection and/or differentiation of any one, two, three, four, five, six, seven, eight, nine, ten, eleven, twelve, or more target analytes.

In some embodiments, results indicating the presence of absence of multiple target nucleotides may be provided from analysis of a single patient sample, such as a nasal or nasopharyngeal swab. For example, in some embodiments, a single sample may be analyzed in a single, disposable and self-contained cartridge for the presence or absence of multiple pathogenic targets in the single sample, such as multiple bacterial, viral, and/or fungal nucleotides, and/or mixtures and combinations of the same. In some embodiments, a single sample may be analyzed with a single cartridge, as described herein, for the presence of multiple viral nucleotides, wherein the viral targets comprise four different viral RNA targets including influenza A, influenza B, RSV, and/or SARS-CoV-2.

In some embodiments, the systems, assays, devices, methods and kits described herein, provide extraction, amplification and detection of viral RNA or DNA present from a sample, such as a nasal, nasopharyngeal, sputum or blood sample, obtained from a symptomatic patient. In some embodiments, the technology provided herein may perform the complete analysis including extraction, amplification and detection in less than less than about 1 hour, less than about 30 minutes, and/or less than about 20 minutes, such as about 22 minutes.

In some embodiments, sample analysis by the systems, assays, devices, methods and kits described herein, is initiated by placing a patient sample, such as a sample collected on a swab placed in nasal passage or in the mouth or throat, in a transport media, such as a viral transport media. In some embodiments, the transport media containing specimen sample extracted from the swab is transferred to a liquid sample addition port, or sample port, of a cartridge as described herein. In other embodiments, the swab is directly inserted into the cartridge for processing of the sample on the swab, as described herein.

In some embodiments, the transport media is transferred to a sample port of a cartridge with a transfer pipette supplied as part of a kit with the cartridge. In some embodiments, the provided transfer pipette includes an overflow chamber and is configured to transfer and/or dispense a specific, fixed and known volume of a sample, such as a patient sample extracted from a nasal or nasopharyngeal swab specimen in a transport media. In some embodiments, the provided transfer pipette is configured to transfer and/or dispense a volume of sample of between about 50-2000 μL, 50-1000 μL, 100-500 μL, 150-400 μL, 175-350 μL, 200-300 μL, 225-275 μL or a sample, or about 150 μL, about 200 μL, about 250 μL, about 300 μL or about 350 μL of a sample.

In some embodiments, after a sample is introduced to the sample port of a cartridge, the port is closed and the cartridge is inserted into an instrument for initiation of sample processing. In some embodiments, as detailed herein, the sample is pushed out of the sample port by a lysis buffer. In some embodiments, the lysis buffer also rehydrates a process control, such as a Escherichia virus MS2 (MS2) process control. In some embodiments, the sample and process control, together with particles or beads, such as magnetic particles, are moved into an extraction chamber of a cartridge as described herein. In some embodiments, the solution comprising the sample, optionally a process control, and a fluid (such as a lysis buffer or transport medium) is mixed in the extraction chamber, and cells or organisms in the sample are further lysed by the mixing. In an embodiment, the mixing is by sonication of the extraction chamber. In some embodiments, beads having sample DNA and/or RNA associated therewith are washed, and the DNA and/or RNA is eluted from the beads. In some embodiments, a solution comprising the purified and/or isolated DNA and/or RNA is used to rehydrate a lyophilized master mix that comprises reagents for amplification of the DNA and/or RNA. In an embodiment, the solution with the isolated and/or purified DNA and/or RNA is moved from the extraction chamber into a plurality of reagent chambers, each reagent chamber in dedicated fluid commutation with a detection chamber. Each reagent chamber comprises reagents for amplification and detection of a particular DNA or RNA target analyte. In this way, a cartridge with 2, 4, 6, 8, 10, 12 or any number of reagent chambers with dedicated detection chambers achieves multiplexing analysis of target nucleic acids from a single sample. In an embodiment, the cartridge comprises four reagent chambers each with a dedicated detection chamber, where each reagent chamber comprises reagents (e.g., primers, probes, enzymes, salts, sugars, etc.) for amplification and detection of a specific target analyte. In one embodiment, each reagent chamber comprises reagents (also referred to in the art as master mixes) for amplification and detection of one of a nucleic acid from a specific pathogen. In an embodiment, the pathogens are selected from influenza A, influenza B, RSV, and SARS-CoV-2. In an embodiment, the pathogens are selected from influenza A, influenza B, RSV, SARS-CoV-2, Bordetella pertussis, Brodetella parapertussis, C. difficile, Group A β-hemolytic Streptococcus (Streptococcus pyogenes), pyogenic Group C/G (Streptococcus dysgalactiae), herpes simplex virus 1, herpes simplex virus 2, varicella-zoster virus, human metapneumovirus, trichomonas, human adenovirus, and parainfluenza virus (PIV-1, PIV-2, and/or PIV-3). In some embodiments, reagent in each reagent chamber is transported into a dedicated detection chamber, where amplification of target nucleic acid sequences can be performed. In some embodiments, amplification in the detection chamber may include Taq-man® multiplex real-time RT-PCR reactions that are carried out under optimized conditions generating amplicons for the targeted virus (if present) and the process control present in the sample. In an embodiment, each detection chamber is configured with an optical window for interrogation by an optics system in the instrument that receives the cartridge, for inspection of the amplicons in the detection chamber to determine presence or absence of particular labels, tags or detection reagents.

In some embodiments, each master mix contains primers and labeled probes, such as dual-labeled probes, unique for one, two, or more viral targets and/or a process control. In some embodiments, the probes are labeled, for example, with a fluorophore on one end and a quencher on the other end. In some embodiments, the master mix reagent comprises reagents for a reverse transcriptase step to produce cDNA of viral RNA, if present, and optimally an MS2 bacteriophage process control RNA. In some embodiments, a polymerase cleaves the probe bound to complementary DNA sequences during DNA amplification, separating the fluorophore from the quencher. In some embodiments, this cleavage generates an increase in fluorescent signal and if sufficient fluorescence is achieved, the sample is reported as positive for the detected target sequence. In some embodiments, the instrument that receives the cartridge also includes a user interface screen for controlling, monitoring and reading results, such as positive, negative, and/or invalid results for the presence of absence of the targeted nucleotide sequences.

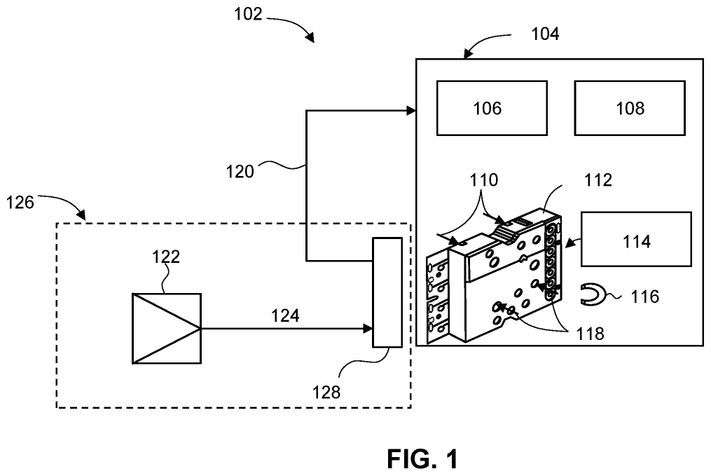

FIG. 1 illustrates a testing architecture 102 that comprises an optical source 122 and an instrument 104 for receiving a cartridge 112, according to some embodiments. The instrument 104 is configured to receive the cartridge 112, wherein the cartridge includes a sample suspected of comprising a target nucleic acid. In some embodiments, the target nucleic acid is part of a nucleic acid of a pathogen, or is associated with a pathogen product or protein. The pathogen may include a bacterium, a virus, a prion, a spore, and the like.

With continuing reference to FIG. 1, the instrument 104 further comprises one or more sonicators, represented in FIG. 1 by sonicators 110, movable in at least one of the x-y-z coordinates. The one or more sonicators are configured to interact with a selected portion or portions of the cartridge 112 (e.g., an extraction chamber, a detection chamber, a sample chamber, combinations of chambers, and the like), for example, to enhance the interaction of components in a chamber and/or to providing mixing for a chemical interaction or reaction there between. In an embodiment, the cartridge comprises a wall that is composed of a flexible material, and the cartridge when inserted into the instrument is positioned so that the one or more sonicators can contact the flexible wall. In an embodiment, the cartridge is composed of a rigid plastic or thermoplastic material and the flexible material is adhered to one side of the rigid cartridge, thereby defining an extraction chamber that has a rigid wall on one side of the cartridge and a flexible wall on an opposing side of the cartridge. A sonicator in the instrument, movable in at least one of the x-y-z coordinates, is movably positioned to sonicate in one or more locations on the flexible wall of the extraction chamber.

In some embodiments, the instrument 104 also includes at least one magnet field 116, such as an electro-magnet, a solenoid, or any element that generates a localized magnetic field, that is movable and/or switchable (on/off). In an embodiment, the localized magnetic field can be positioned to capture a plurality of magnetic beads or particles in the cartridge, such as in the extraction chamber of the cartridge. The magnetic particles may contain, such as adhered to their surfaces, portions of nucleic acid extracted from a cellular or pathogen component in a sample.

In some embodiments, the instrument comprises a pneumatic unit 114 to provide pressurized fluids for moving liquids, such as reagents and eluents, into the cartridge and from one location to another in the cartridge. The pneumatic unit 114 may also use pressurization/de-pressurization to remove reaction residuals from the cartridge. To this end, the pneumatic unit 114 may include a pump, pressurized gas cartridges, or may be coupled with a pressurized air-line or gas-line in a facility. The pneumatic unit 114 can be coupled to the cartridge 112 via a fluid (or gas) manifold (not shown) coupling the fluid sources in the pneumatic unit with valves and conduits in the cartridge. In some embodiments, the instrument 104 includes actuators 118 which mechanically activate components in the cartridge 112, such as pins and other elements to activate piercing elements (e.g., to open reagent canisters in the cartridge), or to open/close valves in fluid conduits and chambers in the cartridge.

With further reference to FIG. 1, in some embodiments, the testing architecture 102 can include an optical unit 126 for illumination of a detection chamber in the cartridge, and for detection of signal therefrom. In some embodiments, the optical unit is part of instrument 104. The optical unit may include an optical source 122 providing an excitation signal 124 delivered to a detection chamber in the cartridge 112 via an optical coupler 128. The optical coupler 128 may also include one or more detectors that provide a response signal 120 from the detection chamber back to a processor circuit 106 in the instrument. The response signal 120 may be an electrical signal transduced from a photosensitive element in the optical coupler 128, indicative of the presence of a suspected target nucleic acid in the sample. In some embodiments, the excitation signal 124 is selected to prompt a desired response signal 120 from the suspected target nucleic acid. For example, in some embodiments, the excitation signal 124 is an optical pump for a fluorescence emission of a tag that has a chemical affinity with at least a portion of the suspected target nucleic acid. Accordingly, the response signal 124 may be provided when the fluorescence emission is detected in the appropriate spectral band of the tag.

With further respect to FIG. 1, the processor circuit 106 executes instructions stored in a memory circuit 108, in the instrument 104. As a result, the instrument 104 may perform at least partially one or more of the steps in methods as disclosed herein. For example, the processor circuit 106 may perform steps for signal processing of the response signal 120. The processor circuit 106 may also control the pneumatic unit 114, the sonicator(s) 110, and the optical unit 126, to perform steps as described in methods disclosed herein.

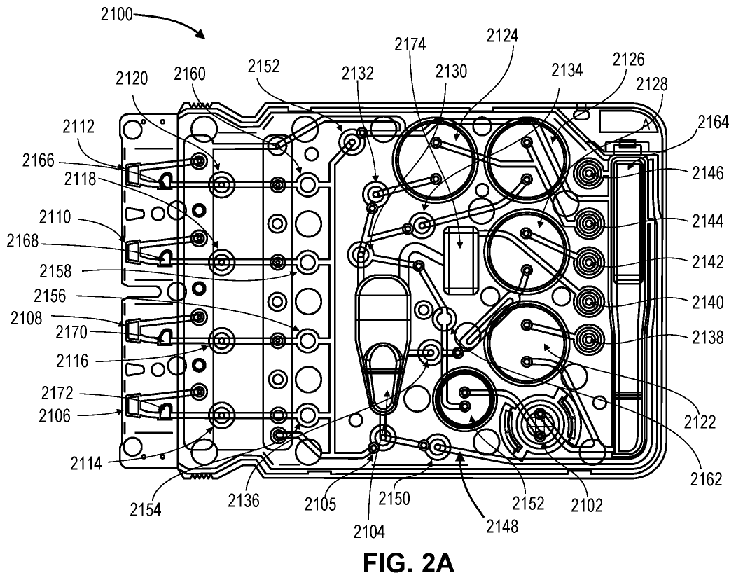

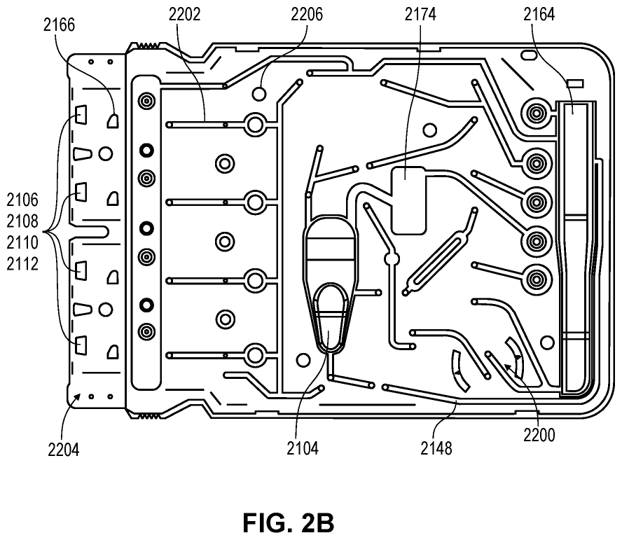

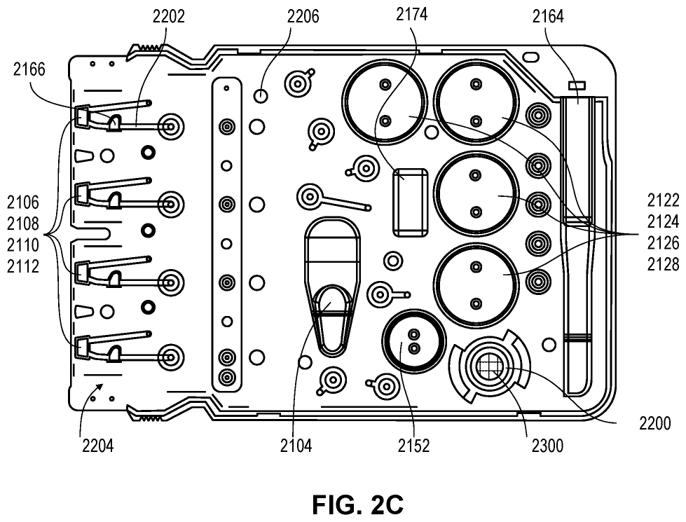

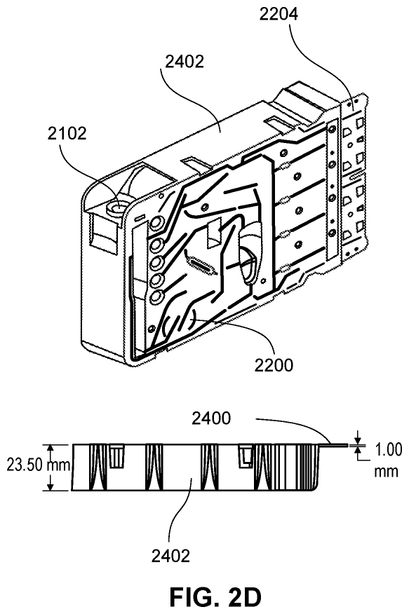

FIGS. 2A-2D illustrate different views of a cartridge for sample testing, according to some embodiments. FIG. 2A illustrates a plan view of a cartridge 2100 for sample testing, according to some embodiments. The cartridge may include a plurality of chambers, including a sample port 2102, an extraction chamber 2104 associated with extraction chamber exit valve 2105, and one or more PCR/detection chambers 2106, 2108, 2110, and 2112 (i.e., in some embodiments a chamber may be configured to serve as both a PCR chamber and a detection chamber), and one or more PCR/detection chamber valves 2114, 2116, 2118, and 2120. In some embodiments, the orientation of the cartridge as it is inserted into the instrument, with the sample port positioned below a midline, provides movement of the sample from the sample port to the extraction chamber in a direction that is opposed to the pull of gravity. In some embodiments, the cartridge may include one or more reagent canisters 2122, 2124, 2126, and 2128, and at least a portion of the reagent canister may be in fluid communication with the extraction chamber, and one or more reagent canister valves 2130, 2132, 2134, and 2136. In some embodiments, the sample port 2102 receives a swab including a sample (e.g., a biological sample). In some embodiments, a second sample port may be configured to receive a liquid sample (e.g., from a drop, or syringe, and the like). In some embodiments, the biological sample includes a body fluid (e.g., blood, serum, plasma, sputum, mucus, saliva, tear, feces, or urine). In some embodiments, the biological sample is human and the presence of one or more portions of a target nucleic acid may indicate a medical diagnostic for an individual providing the sample. Each reagent canister 2122, 2124, 2126, and 2128 contains a reagent that may be lyophilized, or may be in liquid form. In some embodiments, the reagent in at least one of the reagent canisters may include a lysis medium in fluid communication with the sample port through a canister valve 2130, 2132, 2134, and 2136. Once in solution, the reagent reacts with sample components and other reagents, extracting portions of a target nucleic acid that may be present in the sample. Each reagent canister has a volume that may be the same, or approximately the same, as the volume of the extraction chamber.

In some embodiments, the cartridge includes multiple pieces assembled together. A first piece may include an injection-molded plastic piece including the multiple chambers and conduits. Other pieces may include planar covers, or plastic films, having features for alignment of actuators in the instrument and a sample port cover attachment point (cf. small holes and other features). In some embodiments, one or more of the fluid conduits and chambers in the cartridge may include hydrophobic filters to facilitate metering of fluid and complete filling and venting of the detection chambers. In some embodiments, the hydrophobic filters may be welded into the cartridge body. In some embodiments, the cartridge may include one or more bubble traps 2166, 2168, 2170, and 2172 for capturing air bubbles that are forming during the workflow (cf. ‘shark fin’ features). In some embodiments, the action of the sonicators in the instrument occurs against the plastic film.

In some embodiments, the extraction chamber includes a first entry port at a first position and a second entry port at a second, different position. In some embodiments, one of the first entry ports or the second entry port may be located at or below a midline that separates the extraction chamber into two sections of essentially equal volume or into two sections of unequal volume. In some embodiments, the orientation of the cartridge as it is inserted into the instrument, with the sample port positioned below a midline, provides movement of the sample from the sample port to the extraction chamber in a direction that is opposed to the pull of gravity.

With further respect to FIG. 2A, the cartridge can comprise a plurality of gas supply ports, such as ports 2138, 2140, 2142, and 2144, a gas vent 2146, a vent valve 2152, and a waste pathway 2148 fluidically coupled by a waste valve 2150 with a waste chamber 2164 to collect reagent residuals after the reagents are used and cleared from the fluid conduits in the cartridge. In some embodiments, all or a portion of the reagent canisters may be in fluid communication with a dedicated gas source via one or more of the gas supply ports. In some embodiments, the pneumatic unit in the instrument supplies a pressurized gas to move fluids, such as fluids containing the sample or fluidic processing reagents, and the like, through the cartridge, and to remove reaction products or reagent residuals from the extraction or processing chamber into a waste chamber through the waste pathway when a waste valve is open. Gas supply ports 2138, 2140, 2142, and 2144 couple fluid conduits in the cartridge with supply lines in the pneumatic unit. In some embodiments, the pneumatic unit may provide a gas supply to the cartridge via supply lines in the pneumatic unit coupled to gas supply ports of the cartridge. In some embodiments, the pneumatic unit may provide a gas supply to the cartridge at about 2-70 kPa (kilo-Pascals), 2-50 kPa, 2-35 kPa, 5-50 kPa, 5-35 kPa, or 2-150 kPa. In some embodiments, the fluid is a gas, and in some embodiments, the gas may be air, nitrogen, argon, or any other inert gas or combination thereof. The gas vent and the vent valve in the system release gas pressure, and may serve as low pressure points to drive a gas flow from a gas supply port and through one or more chambers in the cartridge.

With continuing reference to FIG. 2A, in some embodiments, the cartridge comprises a canister or a chamber that contains beads or particles, such as magnetic beads or para-magnetic particles (PMPs). In an embodiment, the magnetic particles are introduced into the extraction chamber and, as will be described in more detail below, once introduced into the extraction chamber are retained therein for the remainder of the process. Selected portions of a target nucleic acid from cellular or other pathogenic components in the sample can adhere to the surface of the beads or particles, and in collaboration with the reagents from the reagent canisters provide for isolation and/or purification of target nucleic acid(s), if present, from the sample, as will be further described below.

With further reference to FIG. 2A, in some embodiments, the extraction chamber functions as a metering chamber to receive and/or dispense a precise measure of a liquid. In this embodiment, a liquid, such as an elution medium that is held within a reagent canister or other chamber in the cartridge, is moved into the extraction chamber by a channel or conduit fluidically connecting them. The extraction chamber has a defined, pre-selected volume which, when exceeded, overflows into an overfill chamber 2174, thereby ensuring a defined, metered volume of fluid in the extraction chamber. In an embodiment, the metering function of the extraction chamber is utilized when a fluid, such as an elution medium, is introduced into the extraction chamber. The elution medium is transferred into the reagent chambers and into detection chambers on the cartridge downstream of the extraction chamber, where the nucleic acid(s) in the elution medium is amplified for detection. It is desirable, in some embodiments, to have a known volume of elution medium with nucleic acid placed in each amplification or detection chamber in order to obtain quantitate results. In this embodiment, elution medium is moved into the detection or reaction chambers for nucleic acid amplification via conduits enabled by valves 2114, 2116, 2118, and 2120. In some embodiments, the volume of elution medium in the reagent canister can be larger than the pre-selected volume of the metering chamber. Accordingly, the amount of elution medium transmitted to the extraction chamber is not less than the pre-selected volume of the metering chamber. In embodiments, the volume of elution medium in the reagent canister is greater than the volume of the portion of the extraction chamber that functions as a metering chamber so that elution medium flows into an overfill chamber, thereby ensuring a known, metered volume of elution medium in the extraction chamber. In some embodiments, the overfill chamber 2174 includes a sponge-like material to capture the overflow of media. In some embodiments, a filter paper or other cellulose material may be used as an absorbent of the excess effluent medium. Accordingly, it is desirable to avoid a drip or flow back into the fluid conduits of the cartridge from the overfill chamber 2175. In other embodiments, the overfill chamber 2174 is in fluid communication with the extraction chamber and the liquid medium reagent canisters, permitting a known volume of reagent medium to be introduced into the extraction chamber. In some embodiments, the overfill chamber 2174 is in fluid communication with the extraction chamber 2104 and permits a known volume of liquid media to remain in the extraction chamber. In some embodiments, the volume of the liquid media in at least one of the reagent canisters 2122, 2124, 2126, and 2128 exceeds the volume of the extraction chamber 2104. The excess liquid is removed from the extraction chamber 2104 via an extraction exit valve 2105 and an overflow valve 2152.

With continuing reference to FIG. 2A, cartridge 2100 also comprises chambers comprising a dried or lyophilized master mix (MMX), indicated by identifiers 2154, 2156, 2158, and 2160, and referred to as reagent chambers or PCR reagent chambers. Disposed in the reagent chambers is a bead or lyophilate of amplification reagents (e.g., primers, probes, enzymes, salts, sugars, and the like) for amplification of a target nucleic acid. In some embodiments, a lyophilate within each reagent chamber may be different from the other. Without loss of generality, multiple combinations may be realized, wherein two or more of the reagent chambers can be dedicated to the same lyophilates (e.g., in a 2/2 combination) or three chambers have the same lyophilate (e.g, a 3/1 combination), or each chamber has a lyophilate different from the other. In some embodiments, the reagent chambers may have a bulbous geometry, and the lyophilates may be bead-shaped, or may have any other geometry (e.g., hemisphere, disk, ellipsoidal, and the like), with a diameter smaller than a diameter of the bulbous chamber. In an embodiment, the lyophilate includes reagents for a process control that acts as an internal control for the amplification process. The process control reagent can be, for example, a reagents placed in a chamber of the cartridge, in dried or liquid form, that are moved, generally as a liquid, into the extraction chamber. In some embodiments, the process control reagent can be a lyophilized Escherichia virus MS2 (MS2) bacteriophage process control. In some embodiments, a sonicator in the instrument is positioned for contact with the cartridge to facilitate the mixing of the nucleic acid and the reagents in the PCR reagent chambers. Each reagent chamber is in fluid communication with a detection chamber, where thermocycling can occur and the nucleic acid is amplified. Amplicons in each detection chamber have a detectable label for detection, identification and/or differentiation. The detectable label or tag can be a fluorescence emitting tag, or any other radiation emitting tags attached to the amplicon. Without loss of generality, any number of detection chambers for nucleic acid amplification may be realized in various embodiments.

In some embodiments, a metered volume or quantity of a sample suspected of comprising a target analyte is transferred from a sample chamber via a sample port in conjunction with a liquid, such as a lysis buffer. The liquid can contain or can pass through a chamber with a dried process control, to rehydrate the process control, such as an MS2 bacteriophage process control. In some embodiments, the metered sample together with the process control and magnetic beads, are moved into the extraction chamber. In some embodiments, the solution comprising sample, process control and magnetic beads is mixed by sonication, and cells in the sample are lysed to release nucleic acid into the solution in the extraction chamber. In some embodiments, the lysis buffer is removed from the extraction chamber, with the magnetic beads and any nucleic acid that adheres to the magnetic beads secured in the extraction chamber by a magnetic field applied to the wall of the chamber. One or more different wash fluids can then be sequentially introduced into the extraction chamber and the magnetic beads released into the wash fluid. After the wash steps are completed and wash fluid drained from the extraction chamber, an elution buffer is introduced into the extraction chamber, again with the magnetic beads secured in the extraction chamber by a magnetic field applied to the wall of the chamber. The magnetic beads are released into the elution buffer to elute nucleic acid from the magnetic particles and into the elution buffer. In some embodiments, the elution buffer comprising the nucleic acid isolated from the sample, and optionally the process control nucleic acid, is used to rehydrate the lyophilized master mixes in each PCR reagent chamber.

In some embodiments, the cartridge may include a liquid sample and/or a swab sample presence detection feature(s). Accordingly, when the detects feature indicates presence of a sample in the cartridge and/or a swab in the cartridge, a signal is communicated to the processor circuit in the instrument. Receipt of the signal confirming presence of a sample and/or a swab in the cartridge can initiate a program stored in the instrument to automatically being the sequence of events to determine presence or absence of a target analyte in the sample. In some embodiments, the instrument may provide an alert notification to a user, indicating that the cartridge is ready and able to begin a testing sequence.

The cartridge may include a control sample in a separate chamber or an internal control that is placed in its own dedicated chamber, which may or may not be sent into the extraction chamber for processing. Accordingly, the control sample may include a nucleic acid control that undergoes one or more, or all, of the processing steps of the regular sample, including adhering to the magnetic beads to undergo chemical interactions with all or at least one of the reagents, including the elution medium.

In some embodiments, the cartridge may include insertion features facilitating the instrument to pull the cartridge inside the system, such as a physical fiducial in the form of a tab, notch, divot or the like. In some embodiments, the orientation of the cartridge as it is inserted into the instrument and during processing of a sample is such that the sample port is positioned below a midline intersecting the cartridge in the insertion and processing position. In this embodiment, transfer of the sample from the sample port to the extraction chamber in a direction that is opposed to the pull of gravity.

FIGS. 2B-2D illustrate profiles or configurations of a cartridge for sample testing, according to one embodiment. FIG. 2B illustrates a plan view of a first side of the cartridge, which can be the ‘inner’ side of the cartridge, relative to the instrument. FIG. 2C illustrates a plan view of a second side of the cartridge, wherein the first side and the second side are opposite and complementary to one another. The extraction chamber 2104, the reagent canisters 2122, 2124, 2126, and 2128, the detection chambers 2106, 2108, 2110, and 2112, the waste chamber 2164, the bubble traps 2166, 2168, 2170, and 2172 and the waste pathway 2148 are as described above in relation to FIG. 2A. In the embodiment illustrated in FIGS. 2B-2D, the cartridge comprises two sample ports, a first sample port designed to receive a swab with a sample thereon (2102), and a second sample port (2200) configured to receive a liquid sample, such as by pipette. Hydrophobic filters 2202 may be disposed in the conduits leading to the detection chambers (detection chamber panel 2204) to facilitate fluid flow and avoid residuals to remain in the conduit. Alignment features 2206 enable the positioning of the cartridge within the instrument.

As seen in FIG. 2C the cartridge can comprise an overfill chamber 2174 (optional), and a chamber containing the magnetic beads, 2152. The liquid port 2200 acts as sample port (e.g. sample port in FIG. 2A) for a liquid sample. In some embodiments, the liquid port 2200 may include a frustrated total internal reflection feature 2300 that enables the instrument to detect the presence of a liquid sample (and thus activate the fluid conduits coupled with the liquid port 2200). For example, in some embodiments the port 2200 is designed such that when an optical beam is passed through or across it, if sample is in the beam pathway a detection of liquid is achieved.

FIG. 2D illustrates a perspective view of the cartridge, with the inner side of FIG. 2B in the foreground. Also shown in FIG. 2D is a side view of the cartridge, illustrating the plastic film 2400 covering the inner side of the cartridge (cf. FIG. 2B), and the cartridge body 2402. In some embodiments, the cartridge body is about 20 mm wide (such as 23.5 mm wide) and the plastic film is about 1 mm thick, as also shown in FIG. 2D.

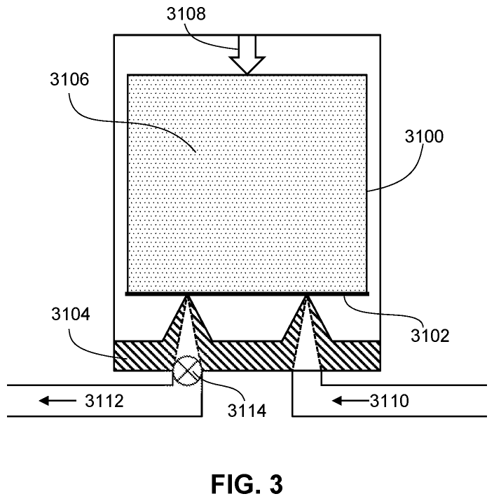

FIG. 3 illustrates a reagent canister 3100 (as exemplary of reagent canisters 2122, 2124, 2126 or 2128 from FIG. 2A or FIG. 2C) and a mechanism for dispensing reagents in the canister into the cartridge, according to some embodiments. In some embodiments, the reagent in all or a portion of the plurality of reagent canisters (such as one of 2122, 2124, 2126 or 2128 from FIG. 2A or FIG. 2C) is a liquid reagent. In some embodiments, the reagent canister 3100 comprises a frangible membrane 3102 and a piercing element 3104 positioned to pierce the frangible membrane 3102. To pierce the frangible membrane 3102, in order to dispense a reagent therein, an actuator 3108 in the instrument that receives the cartridge can press against the canister 3100 causing it to move against the piercing element 3104. The actuator 3108 may include a solid pin pushed against the canister by an electric motor in the instrument. In some embodiments, the piercing element 3104 may be formed (e.g., by injection molding) within the cartridge body; that is, the piercing element 3104 is positioned on the cartridge for contact with and piercing of the frangible membrane 3102 on the reagent canister 3100. In some embodiments, the piercing element may be part of the actuator, part of the instrument, part of the cartridge, or part of the reagent canister. In some embodiments, the frangible membrane or frangible material may be an aluminum laminate, a septum, a valve, a port, or the like (e.g., any element that may be opened upon piercing or puncture, without necessarily rupturing or breaking). In some embodiments, the frangible membrane 3102 may be a laminate of multiple layers of material. A headspace may exist between the piercing element 3104 and the frangible membrane 3102, and bias by actuator 3108 moves the reagent canister into the headspace and into contact with the piercing element. Alternatively, a headspace may exist between the reagent canister and the actuator, with the piercing element in contact or near contact with the frangible material, and movement of the actuator across the headspace to contact the reagent canister results in the piercing element penetrating the frangible material. Once the integrity of the frangible material in disrupted, reagent in the reagent canister can be released.

In some embodiments, the piercing element 3104 may include a hollow conduit fluidically coupling an inlet 3110 with an outlet 3112 once the frangible membrane 3104 is pierced. In some embodiments, a valve 3114 at the inlet or outlet may further control the flow of reagent 3106 out of the canister 3100, once the actuator 3108 has ruptured the frangible membrane 3102. In some embodiments, a gas from a dedicated gas source is movable from the gas source to a reagent canister 3100 via the hollow conduit. In some embodiments, each reagent canister interacts with one actuator in the instrument. In some embodiments, each reagent canister interacts with a dedicated actuator. In some embodiments, when the actuator is not activated and the frangible membrane is intact, the valve 3114 may be opened, allowing a gas flow from the inlet to the outlet through the reagent chamber and the hollowed piercing member. This may be useful to remove residual components and/or reagents in the extraction chamber from a prior step before actuating the piercing mechanism to transfer the reagent solution to the extraction chamber.

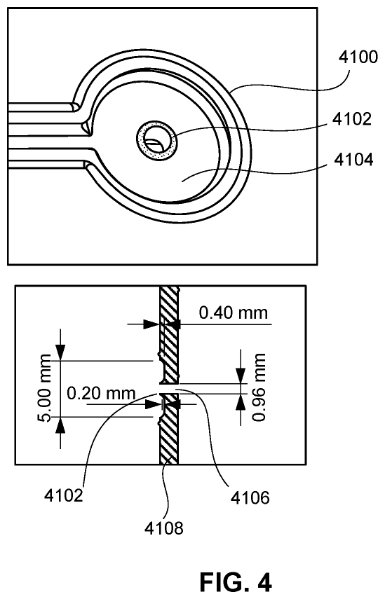

FIG. 4 illustrates a valve used to control fluid flow in the cartridge, according to some embodiments. In some embodiments, the valve is a pinch valve including a film welding energy director 4100, a valve seat 4102, and a valve cavity 4104. A cross-sectional view illustrates some exemplary dimensions, in mm, of the valve seat 4102, the cartridge body 4108, and the valve through-hole 4106. Without loss of generality, the dimensions of the valve may be any dimensions adapted for the specific desires of a given cartridge and instrument.

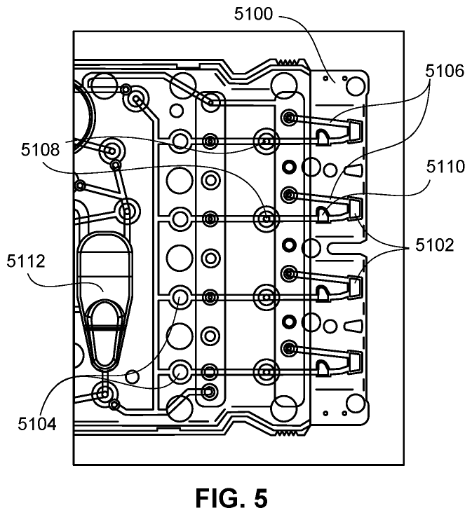

FIG. 5 illustrates a partial view of a detection chamber panel 5100 in a cartridge for sample testing, according to some embodiments. The detection chamber panel comprises a plurality of detection chambers, such as those indicated at 5102, which may be PCR amplification chambers, each fluidically coupled to a reagent chamber, such as PCR reagent chamber 5104. Fluid conduits, such as those indicated at 5106, provide dedicated fluid communication between a detection chamber via a PCR valve, such as valve 5108, and a bubble trap, such as bubble trap 5110. The extraction chamber 5112 in the cartridge is shown in the drawing figure for completeness and to provide perspective.

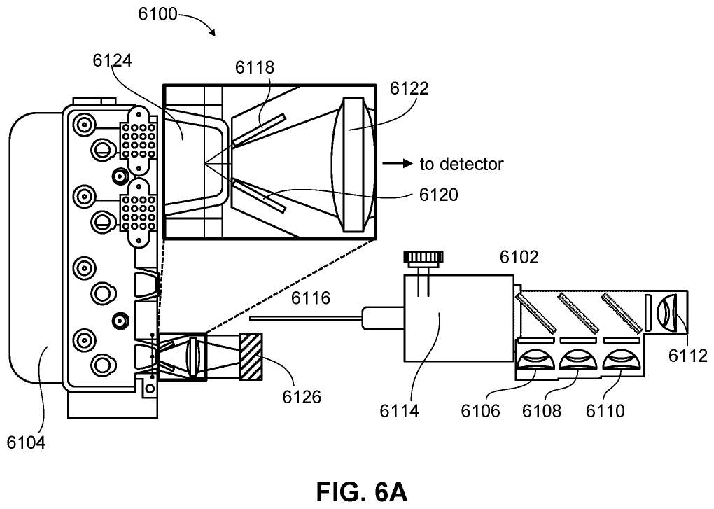

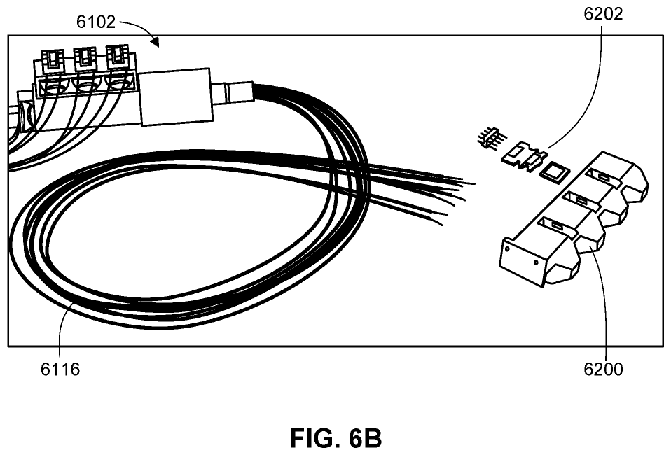

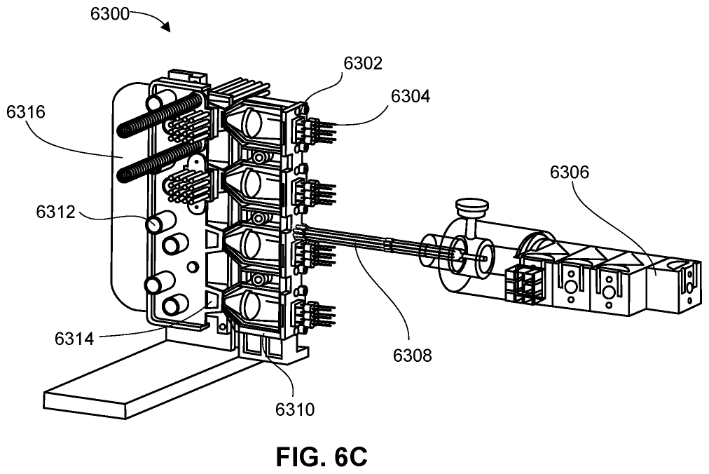

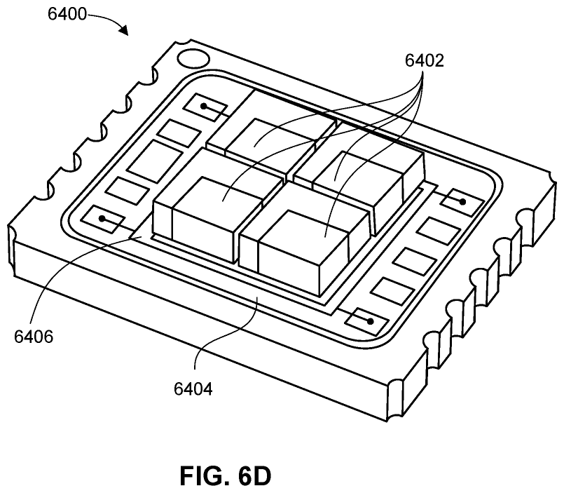

FIGS. 6A-6D illustrate components in an optical coupler 6100 for use in an optical unit for an instrument, according to some embodiments. FIG. 6A illustrates multiple optical sources assembled in an excitation optics setup 6102 including filters, lenses, and optical fibers for delivery of the excitation signal to a detection chamber 6124 in a cartridge as disclosed herein. The optical source may include a set of one or more (e.g., four) light emitting diodes (LEDs), lasers, or any other type of monochromatic, or quasi-monochromatic light sources 6106, 6108, 6110, and 6112. The LEDs may be configured to provide excitation signals for different fluorescence emission tags. Each of the fluorescent emission tags may be indicative of a specific target nucleic acid portion to be detected. In the excitation optic setup illustrated, a filter in front of each LED serves as a band-pass filter for the excitation light to avoid cross talk, and may also include an antireflection (AR) coating to avoid fringing and other etalon effects in the optical module. To combine and multiplex the multiple LEDs used, some embodiments may include a beamsplitter configuration including dichroic or birefringent elements that transmit light in one wavelength or polarization and reflect light in a different wavelength or polarization.

The excitation optics setup also includes a coupler 6114 to couple the light from the LEDs into a bundle of excitation fibers 6116. In some embodiments, and without limitation, the bundle of excitation fibers 6116 may include eight (8) fibers (two for each of the detection chambers). In some embodiments, the excitation optics setup may include fewer optical fibers or more optical fibers. Moreover, in some embodiments, the excitation optics setup may be a free-space excitation optics setup with no optical fibers.

As shown in the figures, two excitation fibers 6118 and 6120 may converge on each detection chamber 6124 in the detection chamber panel 6104. The two excitation fibers converge at an angle relative to one another, and a collection lens 6122 collects the optical signal emitted from the detection chamber 6124. In some embodiments, the collection lens 6122 is disposed between the two excitation fibers such that the collection lens receives a cone of the emitted signal from the sample. In some embodiments, the collection setup is such that the cone of the emitted signal collected by the collection lens has a reduced amount of scattered excitation light from the excitation fibers, and a reduced amount of stray light from the background of the sample port.

The collection lens receives the cone of emitted light from the sample and focuses it on a detector 6126. The detector transduces the optical signal into an electric response signal that may be processed by the processor circuit in the instrument.

FIG. 6B illustrates the excitation optics setup 6102 of FIG. 6A, further including a pick-up component 6200 for coupling with the detection chamber panel in the cartridge, and a detector with connectors 6202 for providing the response signal. The pickup component 6200 may be configured to fit into the detection chamber panel in the cartridge and to receive the bundle including the excitation fibers 6116 carrying the excitation signals. The pickup component 6200 also serves as a mount for the detector and connectors 6202, and may have a shape and size that reduce the amount of stray light reaching the detector.

FIG. 6C illustrates a schematic view of an optical module 6300 coupled to a detection chamber panel 6316 in a cartridge, according to some embodiments. The figure illustrates the elements in an optical module (e.g., detectors 6302, connectors 6304, optical sources 6306, a fiber bundle 6308) as described above, assembled together in a pick-up component 6310. Also illustrated are valve access ports 6312 for actuators in the instrument to access valves in fluid conduits to the detection chambers 6314.

FIG. 6D illustrates a detector set 6400 having a filter assembly 6402 disposed adjacent to a silicon detector 6404, according to some embodiments. In some embodiments, the filter assembly 6402 may include emission filters with optimized coating to reduce internal reflections and other undesired etalon effects. In some embodiments, the filter assembly 6402 may include a channel isolation mask 6406 to prevent light cross-talk between the different filters in the assembly. For example, in some embodiments the channel isolation mask 6406 is placed within the filter assembly 6402 to prevent the cross-talk. It is homogeneous between the filters.

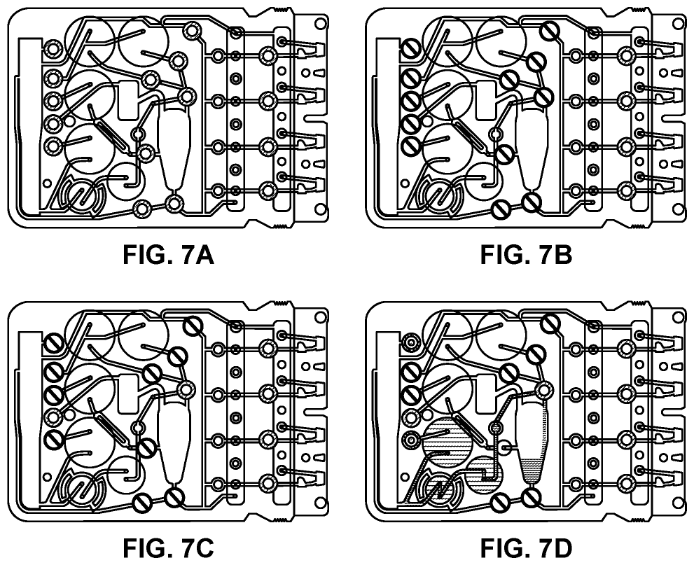

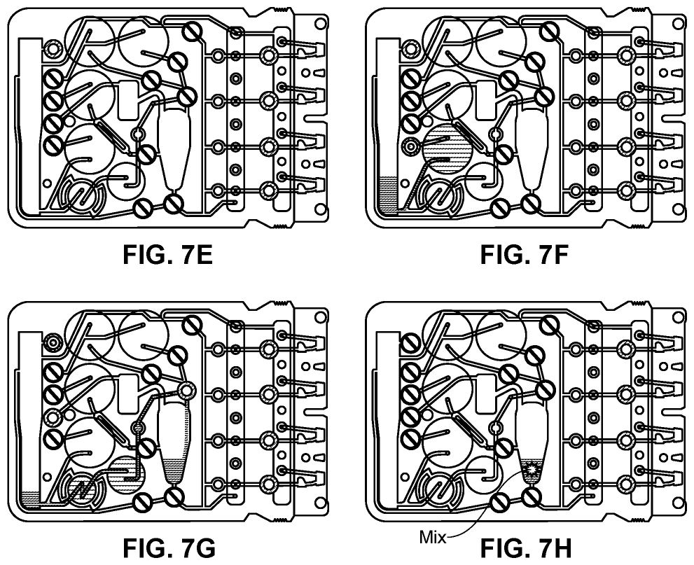

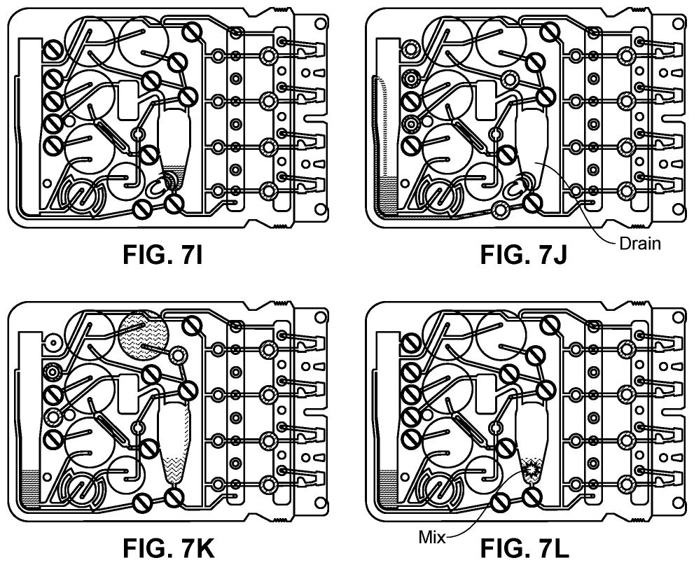

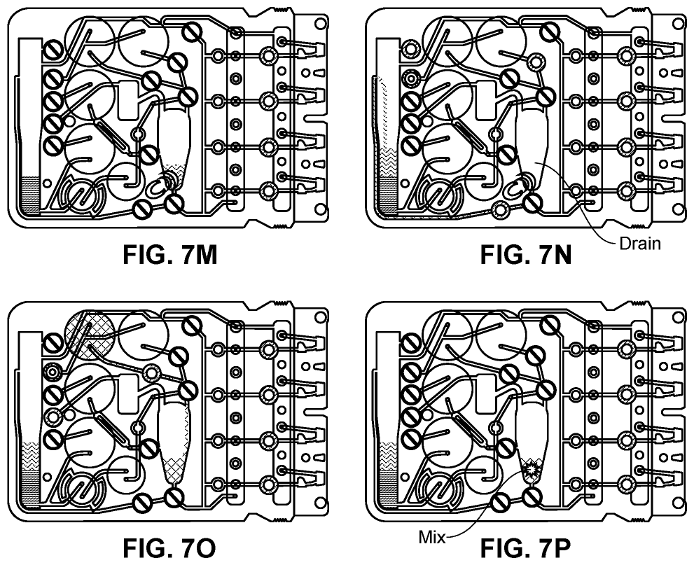

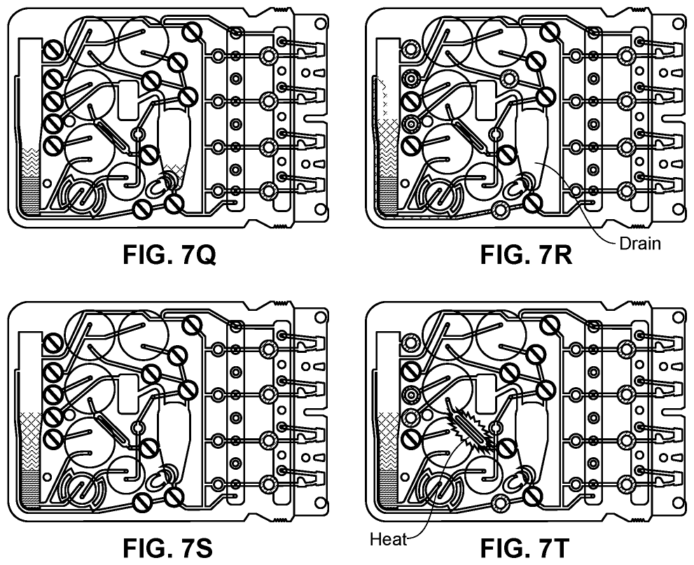

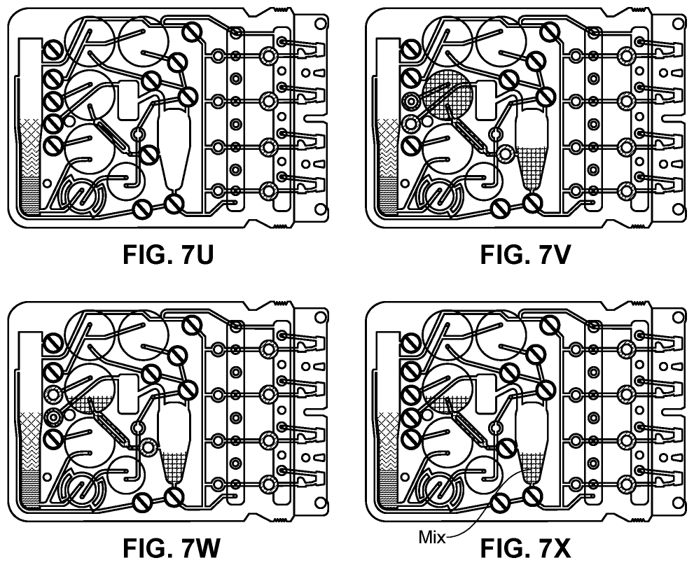

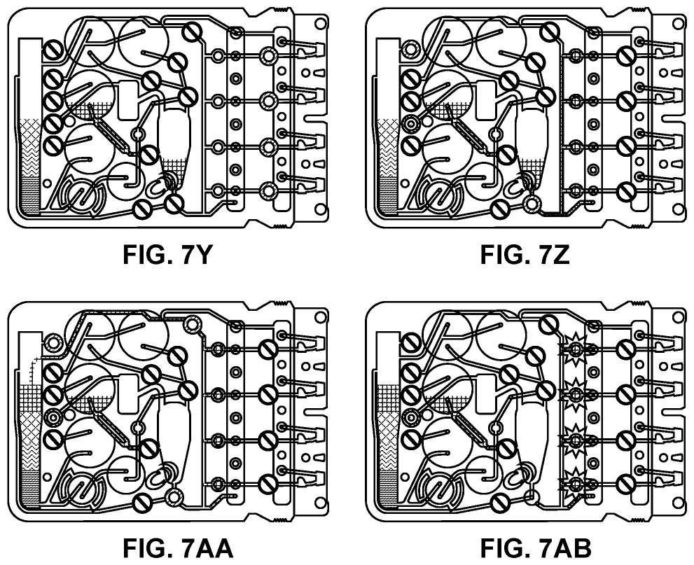

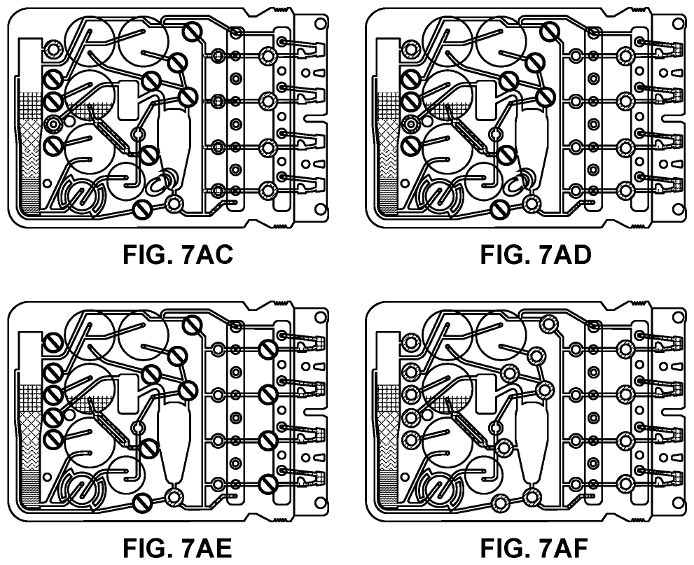

FIGS. 7A-7AF illustrate a sequence of steps in a sample testing method using a cartridge and an instrument, according to some embodiments. Any one of the devices and components in a testing architecture as disclosed herein may perform steps 7A-7AF, at least partially. For example, at least some of the steps in steps 7A-7AF may include inserting a cartridge in an instrument and coupling, with an optical coupler, an excitation signal from an optical source to the cartridge, as disclosed herein (cf. FIG. 1). In an embodiment, the cartridge comprises one or more structural features to facilitate insertion and/or alignment of the cartridge into and with the instrument. Such features can include, for example, a ridge, a hole, a dimple, a divot, a chamfer, and the like. In some embodiments, the orientation of the cartridge as it is inserted into the instrument, with the sample port positioned below a midline intersecting the cartridge, provides movement of the sample from the sample port to the extraction chamber in a direction that is opposed to the pull of gravity.

The optical coupler may include a detector set that generates a response signal when a selected label in the sample receives the excitation signal. The response signal may be received, analyzed and a result for the sample test provided by a processor circuit executing instructions stored in a memory circuit, in the instrument (cf. FIG. 1). The cartridge may include an extraction chamber, a detection chamber, and a sample suspected of including a target nucleic acid. In some embodiments, at least some of the steps may be performed using a pump in a pneumatic system in the instrument, coupled with the cartridge via a gas supply port air manifold (cf. FIGS. 1 and 2). In some embodiments, a method consistent with the present disclosure may include at least one of the steps in steps 7A-7AF, performed in any order. The sequence of steps shown in FIGS. 7A-7AF include various combinations of open and closed valves in order to perform steps 7A-7AF, at least partially, wherein open valves are designated by an open circle and closed valves are indicated by a circle with a line passing through it. Also in FIGS. 7A-7AF, liquid reagent location and movement thereof, and impingement or pushing on various canisters by various actuators, is shown by different shading patterns indicating locations of various liquids as they are moved through features of the cartridge, and locations of various canisters as they are acted upon by various actuators in steps 7A-7AF. Moreover, embodiments consistent with the present disclosure may include one or more of the steps performed simultaneously, quasi-simultaneously, or overlapping in time.

Step 7A comprises placing the cartridge in the instrument.

Step 7B comprises closing all valves in the cartridge, other than each PCR valve positioned between each PCR reagent chamber and each detection chamber.

Step 7C occurs for cartridges with a sample port that receives a liquid sample, and comprises opening certain valves that are associated with the sample port, to prepare for filing the extraction chamber.

Step 7D occurs for cartridges with a sample port that receives a liquid sample, and comprises actuating a force against reagent canister 1, and moving fluid from the reagent canister 1 into the extraction chamber, moving the sample from the sample port into the extraction chamber, moving the PMPs into the extraction chamber. It will be appreciated that the PMPs can be stored in a chamber or canister, or in some embodiments are already positioned in the extraction chamber.

Step 7E occurs for cartridges with a port that receives a swab with a sample, and comprises opening valves in order to permit introducing a fluid from a reagent canister to contact the swab.

Step 7F occurs for cartridges with a port that receives a swab with a sample, and comprises pushing canister 1 to release its fluid into the port that received the swab (swab is not shown in FIG. 7F), and draining canister 1 to the swab port.

Step 7G comprises moving the fluid in contact with the swab into the extraction chamber, where in this embodiment, the fluid moves through a chamber with dried PMPs, and carries the PMPs with the fluid into the extraction chamber. Thus, the sample and the PMPs are now in the extraction chamber.

Step 7H comprises closing all valves, other than the valves between the PCR reagent chambers and the detection chambers, and mixing the components in the extraction chamber (e.g., by actuating the sonicator in the instrument, where the sonicator contacts a surface of the cartridge near or at the extraction chamber).

Step 7I comprises collecting the PMPs (e.g., using a localized magnetic field), typically by gathering the PMPs against a wall of the extraction chamber.

Step 7J comprises draining or removing the fluid in the extraction chamber into the waste chamber through the waste pathway.

Step 7K comprises pushing canister 2 to enable release of its contents, and moving the contents of canister 2 into the extraction chamber. Reagent canister 2, for example, has a wash solution of wash buffer.

Step 7L comprises closing all valves, other than the valves between the PCR reagent chambers and the detection chambers, and mixing the components in the extraction chambers (e.g., by actuating the sonicator).

Step 7M comprises collecting the magnetic beads (referred to in the drawing figure as PMPs, but without intention to be limiting) in the extraction chamber (e.g., by using the localized magnetic field).

Step 7N comprises removing the fluid from the extraction chamber to the waste chamber via the waste pathway.

Step 7O comprises pushing canister 3 to enable release of its contents, and moving the contents of canister 3 into the extraction chamber. Canister 3 can comprise, for example, a wash fluid.

Step 7P comprises closing all valves, other than the valves between the PCR reagent chambers and the detection chambers, and mixing the contents of the extraction chamber (e.g., using the sonicator).

Step 7Q comprises collecting the PMPs (e.g., by using the localized magnetic field), typically by gathering the PMPs against a wall of the extraction chamber.

Step 7R comprises removing the fluid from the extraction chamber into the waste chamber via the waste pathway.

Step 7S comprises closing all valves, other than the valves between the PCR reagent chambers and the detection chambers.

Step 7T comprises opening the valve to open a pathway to reagent canister 4, turning the heater ‘on,’ and pushing heated air through the extraction chamber. In some embodiments, step 7T comprises applying the localized magnetic field to keep the PMPs in the extraction chamber.

Step 7U comprises closing all valves, other than the valves between the PCR reagent chambers and the detection chambers.

Step 7V comprises pushing canister 4 to enable release of its contents, and moving the contents of canister 4 into the extraction chamber. Canister 4 may contain an elution medium.

Step 7W comprises obtaining a defined volume of fluid in the extraction chamber, by using its metering function, where fluid over a certain defined amount exits the extraction chamber at the exit port at approximately the midline of the extraction chamber, and the excess fluid is moved back into canister 4 or into an overflow chamber.

Step 7X comprises closing all valves, other than the valves between the PCR reagent chambers and the detection chambers, and mixing the contents in the extraction chamber (e.g., using the sonicator).

Step 7Y comprises collecting the PMPs (e.g., using the localized magnetic field). The PMPs remain collected in the extraction chamber for the remaining steps in the process.

Step 7Z comprises moving the fluid (usually a defined amount of an elution buffer into which the nucleic acid has eluted from the PMP surfaces) into the PCR reagent chambers and the connecting fluid conduits.

Step 7AA comprises opening the indicated valves to move fluid in the connecting fluid conduits into the waste chamber, to meter and define the volume of fluid in the PCR reagent chambers.

Step 7AB comprises closing all valves, including the valves between the PCR reagent chambers and the detection chambers, and sonicating the PCR reagent chambers.

Step 7AC comprises opening the valves between the PCR reagent chambers and the detection chambers.

Step 7AD comprises moving the fluid in the PCR reagent chambers into the detection chambers.

Step 7AE comprises closing all valves between the PCR reagent chambers and the detection chambers and performing a cycling process in each detection chamber to amplify the nucleic acid. It should be appreciated that any PCR process may be performed in the PCR/detection chambers, including, but not limited to, PCR processes that involve thermal cycling, isothermal reactions, RT-PCR, rapid multiplexed RT-PCR, Taq-man® multiplex real-time RT-PCR reactions, and the like. In some embodiments, PCR processes performed in the PCR/detection chambers assay for the qualitative detection and differentiation of target nucleotides, such as nucleotides from a pathogen of interest. In some embodiments, PCR processes performed in the PCR/detection chambers are carried out under optimized conditions generating amplicons for a nucleotide of interest, such as a targeted viral nucleotide (if present) and any process controls present in the sample.

In some embodiments, step 7AE may also include detection (such as detection, identification, and/or differentiation) of the presence or absence of amplicons of nucleic acids of interest. Such detection may be performed concurrently with, or subsequent to, PCR amplification processes. In some embodiments, detecting amplification products, in the detection chamber, includes probing the detection chamber with an excitation signal from an optical source, via an optical coupler, such as illuminating the detection chamber with the excitation signal and detecting the presence or absence of any signal from amplification targets of interest.

Step 7AF includes disengaging all valves and actuators to enable removing the cartridge form the instrument.

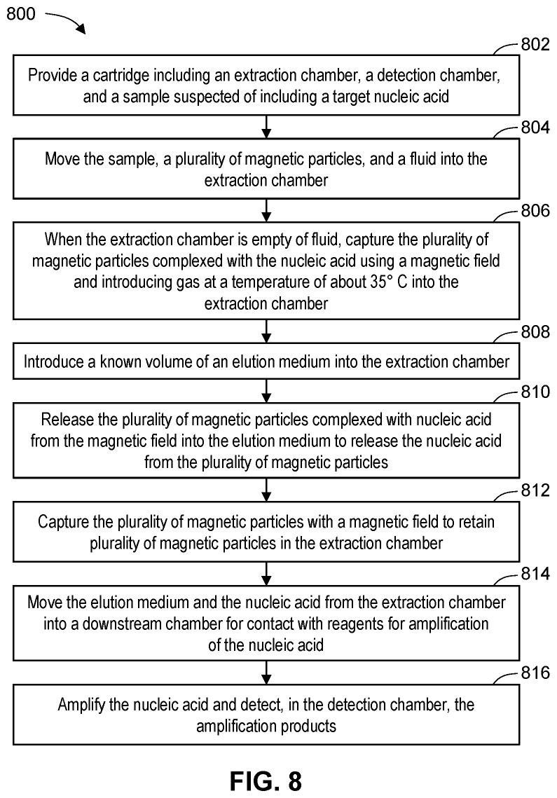

FIG. 8 is a flow chart illustrating steps in a sample testing method 800, according to some embodiments. The method 800 may be performed at least partially by any one of the devices and components in a testing architecture as disclosed herein. For example, at least some of the steps in the method 800 may include inserting a cartridge in an instrument and coupling, with an optical coupler, an excitation signal from an optical source to the cartridge, as disclosed herein (cf. FIG. 1). The optical coupler may include a detector set that generates a response signal when a selected label in the sample receives the excitation signal. The response signal may be received, analyzed, and a result for the sample test provided by a processor circuit executing instructions stored in a memory circuit, in the instrument (cf. FIG. 1). The cartridge may include an extraction chamber, a detection chamber, and a sample suspected of including a target nucleic acid. In some embodiments, at least some of the steps in method 800 may be performed using a pump in a pneumatic system in the instrument, coupled with the cartridge via an gas supply port air manifold (cf. FIGS. 1 and 2). In some embodiments, a method consistent with the present disclosure may include at least one of the steps in method 800, performed in any order. Moreover, embodiments consistent with the present disclosure may include one or more of the steps in method 800 performed simultaneously, quasi-simultaneously, or overlapping in time.

Step 802 includes providing the cartridge including an extraction chamber, a detection chamber, and a sample suspected of including a target nucleic acid. In some embodiments, step 802 includes inserting a swab into a sample port on the cartridge. In some embodiments, step 802 includes rinsing the swab with lysis buffer after insertion into the sample port and prior to moving the sample into the extraction chamber.

Step 804 includes moving the sample, a plurality of magnetic particles, and a fluid into the extraction chamber through a first port of the cartridge. In some embodiments, movement of the sample from the sample port to the extraction chamber in a direction that is opposed to the pull of gravity. In some embodiments, the fluid includes a lysis buffer configured to release a nucleic acid material from a cellular component or a pathogen component in the sample. The pathogen component may be a bacterium, a virus, a prion, a spore, and the like.

Step 806 includes capturing the plurality of magnetic particles complexed with the nucleic acid using a magnetic field when the extraction chamber is essentially or substantially empty of fluid. In some embodiments, step 806 also includes introducing gas at a temperature of above about 35° C. into the extraction chamber.

Step 808 includes introducing a known volume of an elution medium into the extraction chamber through a second port of the cartridge. In some embodiments, the second port of the cartridge is the same as the first port. In some embodiments, the second port of the cartridge is different from the first port of the cartridge.

In some embodiments, step 808 includes heating a region upstream of the extraction chamber and passing a gas through the region to heat the gas and introducing the heated gas into the extraction chamber. In some embodiments, step 808 includes heating the gas at a temperature of above about 35° C. and less than about 90° C. into the extraction chamber.

Step 810 includes releasing the plurality of magnetic particles complexed with nucleic acid from the magnetic field into the elution medium to release the nucleic acid from the plurality of magnetic particles. In some embodiments, step 810 includes moving the lysis buffer and the sample out of the extraction chamber. In some embodiments, step 810 includes moving a wash fluid into the extraction chamber. In some embodiments, step 810 includes releasing the magnetic particles complexed with nucleic acid from the magnetic field into the wash fluid. In some embodiments, step 810 includes sonicating the extraction chamber to release the nucleic acid complexed with the magnetic particles into the elution medium.

Step 812 includes capturing the plurality of magnetic particles with a magnetic field to retain the plurality of magnetic particles in the extraction chamber. In some embodiments, step 812 includes moving the wash fluid out of the extraction chamber. In some embodiments, method 800 includes repeating steps 810 and 812 with a second wash fluid.

Step 814 includes moving the elution medium and the nucleic acid from the extraction chamber into a downstream chamber for contact with reagents for amplification of the nucleic acid. In some embodiments, step 814 is performed through a third port in the cartridge, the third port of the cartridge being different from the first port of the cartridge and the second port of the cartridge. In some embodiments, step 814 includes mixing the elution medium and the nucleic acid with reagents for amplification and detection (such as detection, identification, and/or differentiation) of amplicons of the nucleic acid. In some embodiments, the downstream chamber is a master mix chamber upstream from the detection chamber and step 814 includes sonicating the master mix chamber to form a processing fluid. In some embodiments, step 814 includes heating the detection chamber.

In some embodiments, the detection chamber includes four individual detection chambers, and step 814 includes introducing an amount of the elution medium and the nucleic acid from the extraction chamber into each individual detection chamber.

Step 816 includes amplifying the nucleic acid and detecting, in the detection chamber, the amplification products. In some embodiments, step 816 includes probing the detection chamber with an excitation signal from the optical source, via the optical coupler. In some embodiments, step 816 includes illuminating the detection chamber with the excitation signal.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

In one aspect, a method may be an operation, an instruction, or a function and vice versa. In one aspect, a claim may be amended to include some or all of the words (e.g., instructions, operations, functions, or components) recited in other one or more claims, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

It is claimed:

1. A system, comprising:
a cartridge comprised of:
(i) a plurality of chambers, wherein the plurality of chambers comprises an extraction chamber and a detection chamber;
(ii) a plurality of reagent canisters, each reagent canister in the plurality comprising a reagent;
(iii) a plurality of magnetic particles which once introduced into the extraction chamber are retained therein;
an instrument configured to receive the cartridge, the instrument comprising:
(i) a first sonicator movable in at least one of the x-y-z coordinates;
(ii) a magnetic field positioned to capture the plurality of magnetic particles in the extraction chamber; and
(iii) an optical unit for illumination of the detection chamber and for detection of signal therefrom.

2. The system of claim 1, wherein a portion of the plurality of reagent canisters are in fluid communication with the extraction chamber.

3. The system of claim 1, wherein the reagent in a portion of the plurality of reagent canisters is a liquid reagent.

4. The system of claim 1, wherein all or a portion of the plurality of reagent canisters is in fluid communication with a dedicated gas source.

5. The system of claim 1, wherein one or more reagent canisters in the plurality of reagent canisters includes a frangible membrane and a piercing element.

6. The system of claim 5, wherein the piercing element includes a hollow conduit.

7. The system of claim 6, wherein gas from a dedicated gas source is movable from the gas source to a reagent canister via the hollow conduit.

8. The system of claim 1, wherein each reagent canister interacts with an actuator in the instrument.

9. The system of claim 8, wherein each reagent canister interacts with a dedicated actuator.

10. A cartridge, comprising:
(i) a plurality of chambers, wherein the plurality of chambers comprises an extraction chamber and at least one detection chamber;
(ii) an inlet port for engagement with a gas supply source;
(iii) a plurality of reagent canisters, each reagent canister in the plurality comprising a reagent, each reagent canister comprising a frangible closure and a piercing element with an opening capable of fluid communication; and
(iv) a plurality of magnetic particles in a first location and movable into the extraction chamber.

11. The cartridge of claim 10, wherein the plurality of chambers further comprises one or more sample ports in fluid communication with the extraction chamber.

12. The cartridge of claim 11, wherein the one or more sample ports are configured to receive a swab including a sample.

13. The cartridge of claim 10, wherein the one or more sample ports comprise a liquid port configured to receive a liquid sample.

14. The cartridge of claim 13, wherein the liquid port is configured to identify the presence of a liquid sample by frustrated total internal reflection of a light beam, when a liquid sample is present in the liquid port.

15. The cartridge of claim 10, wherein the plurality of reagent canisters comprises a reagent canister with a lysis medium which is in fluid communication with one or more sample ports.

16. The cartridge of claim 10, wherein the plurality of reagent canisters comprises a reagent canister with a volume of an elution medium which is in fluid communication with the extraction chamber.

17. The cartridge of claim 10, wherein the at least one detection chamber comprises two, three, or four detection chambers.

18. The cartridge of claim 10, wherein the at least one detection chamber is in fluid communication with a reagent chamber including reagents for nucleic acid amplification.

19. The cartridge of claim 17, wherein the at least one detection chamber is in fluid communication with a dedicated bulbous chamber including a lyophilate of the reagents for amplification, and each lyophilate within each dedicated bulbous chamber is different from the other.

20. A cartridge, comprising:
(i) an extraction chamber configured to receive and/or dispense a precise measure of a liquid;
(ii) a plurality of reagent canisters, each reagent canister comprising a reagent; and
(iii) a plurality of magnetic particles in a first location and movable into the extraction chamber, wherein once moved into the extraction chamber, the plurality of magnetic particles are retained therein.

21. A method for identifying presence or absence of a target nucleic acid in a sample, comprising:
i. providing a cartridge including an extraction chamber, a detection chamber disposed downstream from the extraction chamber, and a sample suspected of comprising a target nucleic acid;
ii. moving the sample, a plurality of magnetic particles, and a fluid into the extraction chamber;
iii. with the extraction chamber essentially empty of fluid, capturing the plurality of magnetic particles complexed with the target nucleic acid, if present, with a magnetic field and introducing a gas at a temperature of above about 35° C. into the extraction chamber;
iv. introducing a known volume of an elution medium into the extraction chamber;
v. releasing the plurality of magnetic particles complexed with target nucleic acid, if present, from the magnetic field into the elution medium to release the target nucleic acid from the plurality of magnetic particles;
vi. capturing the plurality of magnetic particles with a magnetic field to retain the plurality of magnetic particles in the extraction chamber;
vii. moving the elution medium and the target nucleic acid from the extraction chamber into a downstream chamber for contact with reagents for amplification of the target nucleic acid; and
viii. amplifying the target nucleic acid and detecting in the detection chamber its amplification products.

22. A kit comprising the cartridge of claim 20, a pipette and user instructions, wherein the pipette comprises an overflow chamber and is configured to dispense a fixed sample volume.
